(19) United States
(12) Patent Application Publication
(10) Pub. No.: US 2010/0010394 A1
(43) Pub. Date: Jan. 14, 2010

(54) Noninvasively low-frequency ultrasonic apparatus for the brain therapy

(75) Inventors: Hao-Li Liu, Tao-Yuan (TW); Wen-Cheng Huang, Tao-Yuan (TW)

Correspondence Address: BACON & THOMAS, PLLC, 625 SLATERS LANE, FOURTH FLOOR, ALEXANDRIA, VA, 22314-1176, US

(73) Assignee: Chang Gung University, Tao-Yuan (TW)

(21) Appl. No.: 12/289,420

(22) Filed: Oct. 28, 2008

(30) Foreign Application Priority Data
Jul. 11, 2008 (TW) 097126210

(51) Int. Cl. A61N 7/00

(52) U.S. Cl. 601/2

(57) ABSTRACT

Ultrasonic energy has been proven that at suitable frequency range (670-kHz to 2-MHz), ultrasound can be focused to a specific target, and the concentrated energy has sufficient high acoustic pressure so that capable of inducing localized blood-brain barrier (BBB) disruption, which has an important implication on noninvasively delivering drug into brain.

### BACKGROUND OF THE INVENTION

1. Field of the Invention

The invention relates to an ultrasonic apparatus, particularly to an ultrasonic apparatus that can emit low-frequency spherical wave.

2. Description of the Prior Art

The conventional focusing-type ultrasonic apparatus can generate focused function by selecting suitable frequency range (670 kHz to 2 MHz). Meantime, the focused area has sufficient high acoustic pressure so that capable of inducing localized blood-brain barrier (BBB) disruption, which is an important implication on noninvasively delivering drug into brain. Although the conventional focusing-type ultrasonic apparatus can induce localized BBB disruption temporarily, yet they are still high-frequency ultrasonic systems. Thus the invasive way has to be used to induce BBB disruption, and it still is concentrated to a small area. It is unable to reach the work of inducing larger area of BBB disruption. Also, the cost of system is pretty high and there are no more extensive applications.

Although the high-frequency ultrasonic system is easier to focus energy to the focal point, most energy is easy to be absorbed by hard tissues such as skull etc, thus the ultrasonic effect will be lost. It can only be operated after the animal skull being removed; thus the high-frequency ultrasonic system is still an invasive drug release brain therapy system.

Due to ultrasonic energy can only focus to a single focal point, it is unable to induce larger area of BBB disruption effectively. Although the low-frequency ultrasound can penetrate through the skull, and can induce larger area of BBB disruption without removing the skull in clinical practice, its shortcoming is that it does not have focusing function, however, it is even unable to focus ultrasound effectively on the animal tissue to be induced.

Therefore, due to the current ultrasonic technique is unable to obtain effective focusing effect and large noninvasive disruption, it is more important to develop the advanced ultrasonic technique to induce BBB disruption.

### SUMMARY OF THE INVENTION.

The feature of the invention is to use the low-frequency plane non-focusing type ultrasound to induce BBB disruption, and use the mechanical, movable way to induce localized BBB disruption temporarily.

Compared to the focusing type ultrasound system, the feature of the invention is that this system can expand the inducing area of BBB disruption effectively.

In addition, due to the frequency used in this system is much lower than the existing technique, the shielding phenomena caused by the skull's absorption of ultrasound can be reduced. The noninvasive therapy can be conducted without removing the skull.

There are advantages for the system further including low cost, simple and easy system, high mobility and very big design novelty.

The advantage for the system is that it is a simple, easy, and portable apparatus without complicated driving equipment.

In addition, the invention can generate the spherical wave by the ultrasonic energy, and high acoustic pressure can be used to induce BBB disruption. Due to the influence range caused by the spherical wave is quite large, and then it will be favorable for the therapy in a larger area. Meantime, the invention can also use filter and mechanical movement to obtain a localized focusing effect upon small area therapy. Thus, there is the biggest therapy flexibility for the design of the system. Furthermore, compared with the conventional high-frequency ultrasonic system, the low-frequency plane non-focusing type ultrasound will be better in drug releasing for the large area.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 shows a preferred embodiment of the invention.

FIGS. 2A and 2B show the installation way of the ultrasonic transducer of the invention.

FIGS. 3A, 3B and 3C show the hollow filter plate with different design.

FIG. 4 shows that the effect by the manual-type focusing for inducing BBB disruption (a) wavelength 50 ms, non-focusing; (b); wavelength=100 ms, non-focusing; (c) wavelength=100 ms, manual-type focusing; (d) wavelength=100 ms, manual-type focusing.

FIG. 5 shows the observation result of BBB disruption versus time.

### DESCRIPTION OF THE PREFERRED EMBODIMENT

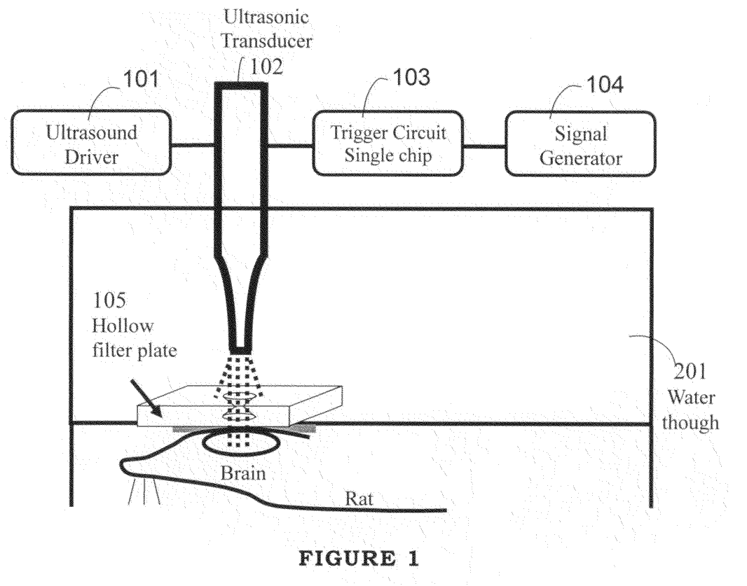

The invention relates to an ultrasonic apparatus having low-frequency spherical wave. As shown in FIG. 1, it comprises the ultrasound driver 101 has driving function, which can drive the ultrasonic transducer 102; the ultrasonic transducer 102, which can generate low-frequency ultrasound as 28 kHz. The trigger circuit single chip 103 has circuit control function. The signal generator 104 can generate sine burst signal, and the hollow filter plate 105 has ultrasound filtering function.

As shown in FIG. 1 again, the ultrasound driver 101 is connected to the ultrasonic transducer 102. The trigger circuit single chip 103 is connected to the signal generator 104 and the ultrasonic transducer 102. The hollow filter plate 105 is also called as the focusing device having filtering and focusing function for ultrasound. It is installed at the tip of the ultrasonic transducer 102, which is the low-frequency device of the invention.

The invention must be installed in water trough 201 while it is under using. The signal generator 104 generates the sinusoidal-wave burst signal. The trigger circuit single chip 103 emits the circuit signal, to trigger the ultrasonic transducer 102. The ultrasound emitted by the ultrasonic transducer 102 can be delivered to a specific target of animal tissue through the hollow filter plate 105.

The invention uses a higher MI value which is easier to be reached by low-frequency ultrasound as MI=P/\sqrt{f}, where P is the acoustic pressure (MPa), f is the frequency (MHz). Therefore the spherical wave can be generated to obtain the same result. The invention uses the hollow filter plate 105 to control the release of ultrasound. The different focusing results can be obtained through various hollow filter plates, in order to induce localized BBB disruption of animal's brain without complete destruction.

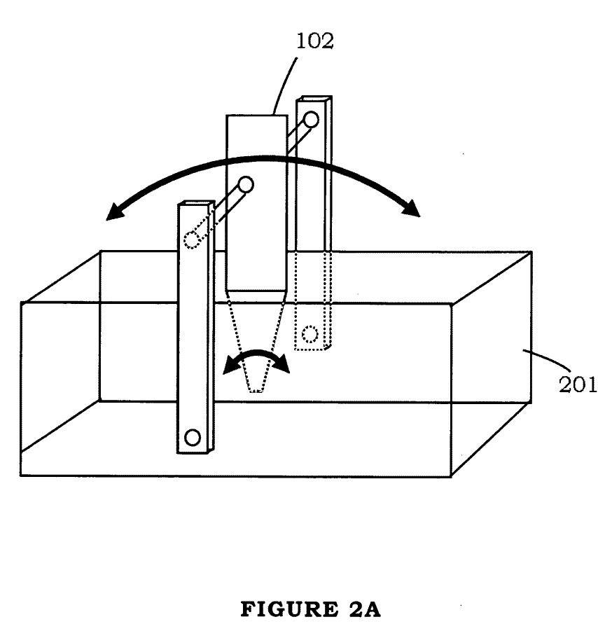

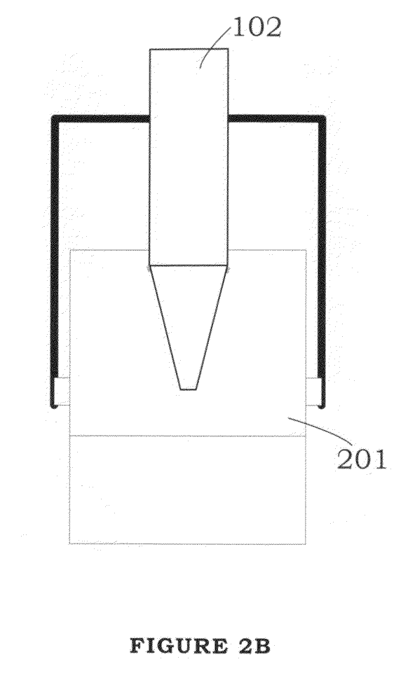

As shown in FIG. 2A, the ultrasonic transducer 102 can adjust the angle, for example, turning to the left or turning to the right.

As shown in FIG. 2B, the ultrasonic transducer 102 with constant angle is shown.

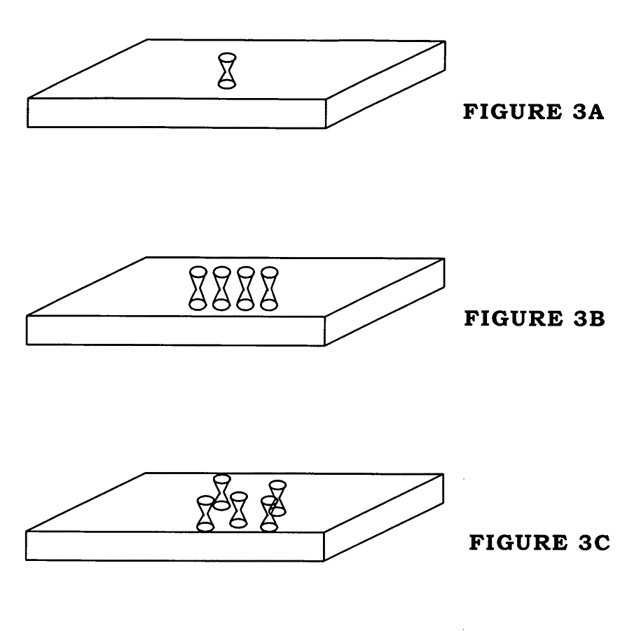

As shown in FIGS. 3A, 3B and 3C, the device having different hollow filter plate can emit the ultrasound at the different angle. It can improve the feature of un-focusing for low-frequency ultrasound; also the low-frequency ultrasound can focus on the same point.

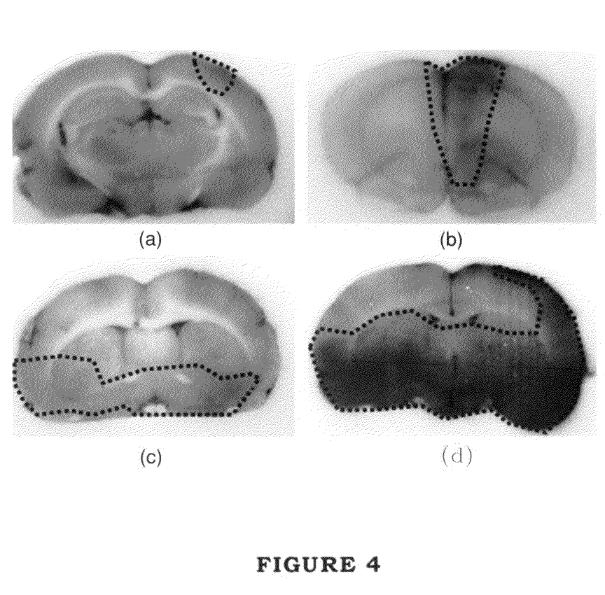

For increasing potential of the system, the low-frequency ultrasound focusing is carried out by the manual type. By experiment comparison results, there are all under the same time parameter, FIG. 4(a), FIG. 4(b) individually shows the difference between the non-focusing and the manual-type focusing under the wavelength=50 ms and FIG. 4(c), FIG. 4(d) individually shows the difference between the non-focusing and the manual-type focusing under the wavelength=100 ms. By observing the results, the lower row figures shown by the manual-type focusing, the BBB disruption is induced in the deep area, i.e. the manual-type focusing could transfer the energy to the deep area of the brain in order to achieve the deep focusing function. In the upper row figures without focusing, the BBB disruption is induced and focused in the shallow surface area of the brain, i.e. the ultrasound energy is dispersedly giving so that there is no more effect for curing the deep area. Therefore, the system can achieve the function for inducing the BBB disruption of the deep area after the manual-type focusing.

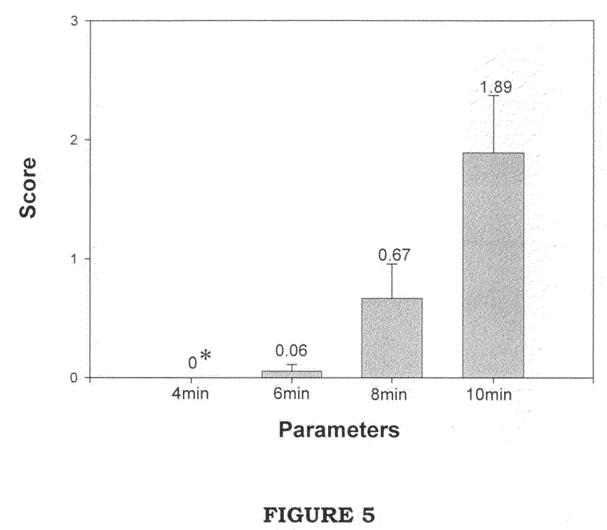

As shown in FIG. 5, the observation result of BBB disruption versus time is shown. It is shown that BBB disruption degree is changed with ultrasound therapy time. The shorter time is, the smaller area is induced. When the therapy time is shortened to 4 minutes, the phenomenon of BBB disruption is unable to be observed. Therefore, it can be concluded that the critical value of inducing BBB disruption should be located between 4 minutes to 6 minutes.

From the above-mentioned description, it is known that the low-frequency apparatus can be used to induce BBB disruption. The noninvasive therapy can be conducted without removing the skull. Therefore the large area of BBB disruption can be induced for the brain therapy.

In addition, compared to the high-frequency ultrasonic system, the shortcoming of high-frequency ultrasound is unable to directly penetrate the skull, so that the low-frequency ultrasound can increase the penetration for inducing noninvasively BBB disruption and it is a kind of noninvasive. The invention provides an ultrasonic apparatus, which uses the low-frequency non-focusing type ultrasound to penetrate animal's skull, in order to induce localized BBB disruption noninvasively.

It is understood that various other modifications will be apparent to and can be readily made by those skilled in the art without departing from the scope and spirit of this invention. Accordingly, it is not intended that the scope of the claims appended hereto be limited to the description as set forth herein, but rather that the claims be construed as encompassing all the features of patentable novelty that reside in the invention, including all features that would be treated as equivalents thereof by those skilled in the art to which this invention pertains.

What is claimed is:

1. A noninvasively low-frequency ultrasonic apparatus, comprising:
an ultrasound driver having driving function;
an ultrasonic transducer for generating a low-frequency ultrasound;
a trigger circuit single chip having circuit control function;
a signal generator for sinusoidal-wave burst signal; and,
a focusing device having filtering and focusing function of ultrasound; wherein the ultrasound driver being connected to the ultrasonic transducer, the trigger circuit single chip being connected to the signal generator, the signal generator being connected to the ultrasonic transducer, the focusing device is installed at the tip of the ultrasonic transducer to form the noninvasively low-frequency ultrasonic apparatus.

2. The apparatus according to claim 1, wherein the frequency of low-frequency ultrasound is about 28 kHz.

3. The apparatus according to claim 1, wherein the angle of ultrasonic transducer can be adjusted.

4. The apparatus according to claim 1, wherein the focusing device comprises a hollow filter plate.

5. The apparatus according to claim 4, wherein the focusing device comprises filtering and focusing function for ultrasound.

6. The application method for a noninvasively low-frequency ultrasonic apparatus, comprising:
generating a signal to a trigger circuit single chip, the signal being generated by a signal generator;
emitting a trigger circuit signal to an ultrasound driver, the trigger circuit signal being generated by a trigger circuit single chip;
driving the ultrasonic transducer by the ultrasound driver, so that the ultrasonic transducer being able to emit a low-frequency ultrasound;
focusing the low-frequency ultrasound, delivering low-frequency ultrasound by the focusing device to achieve the application method of the noninvasively low-frequency ultrasonic apparatus.

7. The application method according to claim 6, wherein the method further comprises it is used in water trough.

8. The application method according to claim 6, wherein the angle of ultrasonic transducer is adjusted.

9. The application method according to claim 6, wherein the focusing device comprises a hollow filter plate.
